(19) United States
(12) Patent Application Publication
(10) Pub. No.: US 2026/0179217 A1
(43) Pub. Date: Jun. 25, 2026

(54) APPARATUS AND METHOD FOR DIAGNOSING EMBRYO IMPLANTATION POTENTIAL USING ARTIFICIAL INTELLIGENCE-BASED DIAGNOSTIC MODEL

(71) Applicant: KNU-Industry Cooperation Foundation, Chuncheon-si (KR)

(72) Inventors: Sanghee LEE, Chuncheon-si (KR); Hayoung LEE, Chuncheon-si (KR)

(73) Assignee: KNU-Industry Cooperation Foundation, Chuncheon-si (KR)

(21) Appl. No.: 19/036,420

(22) Filed: Jan. 24, 2025

(30) Foreign Application Priority Data
Dec. 19, 2024 (KR) 10-2024-0191115

(51) Int. Cl. G06T 7/00; G06T 7/70; G16H 30/40; G16H 50/30

(52) U.S. Cl. CPC G06T 7/0012; G06T 7/70; G16H 30/40; G16H 50/30; G06T 2207/20081; G06T 2207/20084; G06T 2207/30044

(57) ABSTRACT

An apparatus for diagnosing an embryo implantation potential using an artificial intelligence-based diagnostic model according to the present disclosure includes a memory storing a diagnostic model trained to determine an implantation potential index for an embryo based on an image generated by photographing one or more embryos formed by fertilization of a sperm and an oocyte and before implantation, and a processor for inputting a diagnostic image generated by photographing one or more first embryos, which are determination targets of the implantation potential index, into the diagnostic model as input data, and receiving implantation potential index data, which is a result of determining the implantation potential index for the first embryo, as output data from the diagnostic model.

### CROSS-REFERENCE TO RELATED APPLICATION(S)

This application claims the benefit under 35 USC 119(a) of Korean Patent Application No. 10-2024-0191115, filed with the Korean Intellectual Property Office on Dec. 19, 2024, the disclosure of which is incorporated herein by reference in its entirety.

### TECHNICAL FIELD

The present disclosure relates to an apparatus and method for diagnosing an embryo implantation potential using an artificial intelligence-based diagnostic model, and more specifically, to an apparatus and method for diagnosing an embryo implantation potential using an artificial intelligence-based diagnostic model by inputting an image generated by photographing at least one first embryo into a diagnostic model trained to diagnose an implantation potential index including at least one of a development stage of an embryo, a morphological quality of the embryo, and an implantation-related gene expression level of the embryo, and receiving implantation potential index data, which is the result of diagnosing the implantation potential index for the first embryo, from the diagnostic model, thereby quickly and accurately diagnosing implantation ability of the embryo.

### BACKGROUND

Elderly infertile patients have a relatively high rate of producing aneuploid embryos and a low rate of producing healthy embryos. Recently, interest in the development of technologies related to embryo selection has been increasing due to the increase in elderly infertile patients.

In order to increase the implantation efficiency of embryos, it is necessary to select the best embryos, create an endometrial environment suitable for implantation, and adjust the appropriate implantation timing for each patient.

Techniques for evaluating embryo quality were introduced in earnest in advanced countries after 2008, and in Korea after 2012.

Among them, a time-lapse method for diagnosing embryos in a non-invasive manner is produced and sold by five representative companies (Vitrolife, ESCO, Astec, Genea BIOMEDX, and Auxogyn), although there are some differences in the performance of each device, and hospitals that have introduced these devices are actively providing evaluation services for embryo morphological changes.

A preimplantation genetic screening (PGS), which diagnoses using an invasive method, was introduced after the success of 24sure single cell screening technology in 2009, and a next generation sequencing (NGS) method was used for preimplantation embryo diagnosis in 2013, illustrating successful results.

Two representative companies (iGenomix, Reprogenetics) are leading the preimplantation genetic screening and diagnosis market.

In addition, mitochondrial DNA (Mt DNA) analysis service in embryos is also added and used for diagnosis.

These two companies occupy most of the market in Europe and the United States, but in Korea, they are providing exclusive services using microarrays produced by Cancerop.

However, this is an invasive method that removes embryo cells, which may reduce the quality of embryos.

Research using substances secreted from embryos is being conducted in various ways to improve or evaluate embryo quality by measuring proteins (for example, histocompatibility antigen class G (HLA-G)) or metabolites (for example, lacate).

Both methods are currently in the research stage or have some technical limitations, and since the equipment sold for analysis is expensive, related services are also expensive, so there is a disadvantage in that high-quality methods cannot be introduced to relatively many patients at low costs.

The metabolic function of mammalian embryos differs depending on the embryo development stage and may be divided into embryos in the division stage immediately after fertilization (preimplantation embryos) and embryos after implantation. After the fertilization, the newly formed embryonic genome is activated, embryo transcription occurs, and the corresponding proteins increase, so the genetic program regulated by maternal transcripts/proteins receives embryo's genetic information and is expressed. Afterwards, the embryonic genome divides into 8 cells and progresses to a morula stage through a compaction process.

Characteristically, more than 50% of the glucose consumed in the blastocyst is not oxidized but converted to lactate. The lactate formed at this time lowers the pH around the embryo, disaggregates uterine tissue, and creates an environment in which the trophoblast can implant.

In addition, the lactate acts as a cell signaling molecule to increase the recruitment of vascular endothelial growth factor (VEGF) to endometrial cells, thereby promoting angiogenesis.

By utilizing these characteristics according to the development stage of the embryo, a non-invasive, highly accurate, short-time, and low-cost embryo diagnosis method is required.

### SUMMARY

An object of the present disclosure is to provide an apparatus and method for diagnosing an embryo implantation potential using an artificial intelligence-based diagnostic model capable of quickly and accurately diagnosing an implantation ability of an embryo by inputting an image generated by photographing one or more first embryos into a diagnostic model trained to diagnose an implantation potential index including one or more of a development stage of the embryo, a morphological quality of the embryo, and an implantation-related gene expression level of the embryo, and receiving implantation potential index data, which is the result of diagnosing the implantation potential index for the first embryo, from the diagnostic model.

The purposes of the present disclosure are not limited to the purposes mentioned above, and other purposes and advantages of the present disclosure that are not mentioned can be understood by the following explanation and will be more clearly understood by the embodiments of the present disclosure. In addition, it will be easily understood that the purposes and advantages of the present disclosure can be realized by the means and combinations thereof indicated in the scope of the patent claims.

An apparatus for diagnosing an embryo implantation potential using an artificial intelligence-based diagnostic model according to the present disclosure, includes: a memory storing a diagnostic model trained to determine an implantation potential index for an embryo based on an image generated by photographing one or more embryos formed by fertilization of a sperm and an oocyte and before implantation; and a processor for inputting a diagnostic image generated by photographing one or more first embryos, which are determination targets of the implantation potential index, into the diagnostic model as input data, and receiving implantation potential index data, which is a result of determining the implantation potential index for the first embryo, as output data from the diagnostic model.

Preferably, the diagnostic model may be trained using training data including a learning image generated by photographing one or more second embryos as samples and training implantation potential index data, which is a result of pre-determining the implantation potential index for the second embryo.

Preferably, the implantation potential index may include one or more of a development stage of the embryo, a morphological quality of the embryo, and an implantation-related gene expression level of the embryo.

Preferably, the diagnostic model may be trained to identify an embryo image indicating the embryo in the image and detect an embryo position which is a position of the embryo image, and the processor may input the diagnostic image generated by photographing the first embryo into the diagnostic model as input data, and receive embryo position data, which is s result of detecting an embryo position, which is a position of the embryo image indicating the first embryo in the diagnostic image, from the diagnostic model as output data.

Preferably, the processor may set an index display position where the implantation potential index indicated by the implantation potential index data is a position displayed in the diagnostic image, based on the embryo position indicated by the embryo position data, and correct the diagnostic image so that the implantation potential index is displayed at the index display position.

Preferably, the processor may set an embryo identification image position which is a position where an embryo identification image for identifying the embryo image indicating the first embryo image in the diagnostic image is displayed in the diagnostic image, based on the embryo position indicated by the embryo position data, and correct the diagnostic image so that the embryo identification image is displayed at the embryo identification image position.

A method for diagnosing an embryo implantation potential using an artificial intelligence-based diagnostic model according to the present disclosure includes: storing, in a memory, a diagnostic model trained to determine an implantation potential index for an embryo based on an image generated by photographing one or more embryos formed by fertilization of a sperm and an oocyte and before implantation; and inputting a diagnostic image generated by photographing one or more first embryos, which are determination targets of the implantation potential index, into the diagnostic model as input data, and receiving implantation potential index data, which is a result of determining the implantation potential index for the first embryo, as output data from the diagnostic model, using a processor.

The method for diagnosing an embryo implantation potential using an artificial intelligence-based diagnostic model according to the present disclosure may further include training the diagnostic model using training data including a learning image generated by photographing one or more second embryos as samples and training implantation potential index data, which is a result of pre-determining the implantation potential index for the second embryo.

Preferably, the implantation potential index may include one or more of a development stage of the embryo, a morphological quality of the embryo, and an implantation-related gene expression level of the embryo.

The method for diagnosing an embryo implantation potential using an artificial intelligence-based diagnostic model according to the present disclosure may further include: training the diagnostic model to identify an embryo image indicating the embryo in the image and detect an embryo position which is a position of the embryo image, and inputting the diagnostic image generated by photographing the first embryo into the diagnostic model as input data, and receiving embryo position data, which is s result of detecting an embryo position, which is a position of the embryo image indicating the first embryo in the diagnostic image, from the diagnostic model as output data, using the processor.

The method for diagnosing an embryo implantation potential using an artificial intelligence-based diagnostic model according to the present disclosure may further include: setting, using the processor, an index display position where the implantation potential index indicated by the implantation potential index data is a position displayed in the diagnostic image, based on the embryo position indicated by the embryo position data; and correcting, using the processor, the diagnostic image so that the implantation potential index is displayed at the index display position.

The method for diagnosing an embryo implantation potential using an artificial intelligence-based diagnostic model according to the present disclosure may further include: setting, using the processor, an embryo identification image position which is a position where an embryo identification image for identifying the embryo image indicating the first embryo image in the diagnostic image is displayed in the diagnostic image, based on the embryo position indicated by the embryo position data; and correcting, using the processor, the diagnostic image so that the embryo identification image is displayed at the embryo identification image position.

Other specific details of the present disclosure are included in the detailed description and drawings.

According to the apparatus and method for diagnosing an embryo implantation potential using an artificial intelligence-based diagnostic model of the present disclosure, it is possible to quickly and accurately diagnose an implantation ability of an embryo by inputting an image generated by photographing one or more first embryos into a diagnostic model trained to diagnose an implantation potential index including one or more of a development stage of the embryo, a morphological quality of the embryo, and an implantation-related gene expression level of the embryo, and receiving implantation potential index data, which is the result of diagnosing the implantation potential index for the first embryo, from the diagnostic model.

The effects of the present disclosure are not limited to the effects mentioned above, and other effects not mentioned can be clearly understood by those skilled in the art from the description below.

### BRIEF DESCRIPTION OF THE DRAWINGS

FIG. 1 is a connection configuration diagram between an apparatus for diagnosing an embryo implantation potential using an artificial intelligence-based diagnostic model and an embryo culture photographing device according to one embodiment of the present disclosure.

FIG. 2 is a block diagram of the apparatus for diagnosing an embryo implantation potential using an artificial intelligence-based diagnostic model according to one embodiment of the present disclosure.

FIG. 3 is a flowchart of a method for diagnosing an embryo implantation potential using an artificial intelligence-based diagnostic model according to one embodiment of the present disclosure.

FIG. 4 is a diagram illustrating examples of a development stage of an embryo and a morphological quality of the embryo used in the apparatus and method for diagnosing an embryo implantation potential using an artificial intelligence-based diagnostic model according to one embodiment of the present disclosure.

FIG. 5 is a diagram illustrating examples of embryo implantation-related genes used in the apparatus and method for diagnosing an embryo implantation potential using an artificial intelligence-based diagnostic model according to one embodiment of the present disclosure.

FIG. 6 is a diagram illustrating examples of training data used in the apparatus and method for diagnosing an embryo implantation potential using an artificial intelligence-based diagnostic model according to one embodiment of the present disclosure.

FIG. 7 is a diagram for explaining a process of training a diagnostic model in the apparatus and method for diagnosing an embryo implantation potential and d using an artificial intelligence-based diagnostic model according to one embodiment of the present disclosure.

FIG. 8 is a diagram for explaining a process of acquiring diagnostic ability index data and embryo position data for each of the first embryos whose implantation potential index and embryo position are not determined in the apparatus and method for diagnosing an embryo implantation potential using an artificial intelligence-based diagnostic model according to one embodiment of the present disclosure.

FIG. 9 is a diagram illustrating examples of the diagnostic ability index data and embryo position data acquired by the apparatus and method for diagnosing an embryo implantation potential using an artificial intelligence-based diagnostic model according to one embodiment of the present disclosure.

FIG. 10 is an example of a diagnostic image corrected to display an embryo identification image and an implantation potential index by the apparatus and method for diagnosing an embryo implantation potential using an artificial intelligence-based diagnostic model according to one embodiment of the present disclosure.

FIG. 11 is another example of the diagnostic image corrected to display the embryo identification image and the implantation potential index by the apparatus and method for diagnosing an embryo implantation potential using an artificial intelligence-based diagnostic model according to one embodiment of the present disclosure.

### DETAILED DESCRIPTION

The advantages and features of the present disclosure, and the method for achieving them, will become clear with reference to the embodiments described in detail below together with the attached drawings. However, the present disclosure is not limited to the embodiments disclosed below and may be implemented in various different forms, and the embodiments are provided only to make the present disclosure complete and to fully inform a person skilled in the art to which the present disclosure belongs of the scope of the present disclosure, and the present disclosure is defined only by the scope of the claims.

The terms used in this specification are for the purpose of describing the embodiments and are not intended to limit the present disclosure. In this specification, the singular also includes the plural unless specifically stated in the phrase. The terms “comprises” and/or “comprising” as used in the specification do not exclude the presence or addition of one or more other components in addition to the mentioned components. The same drawing reference numerals refer to the same components throughout the specification, and “and/or” includes each and every combination of one or more of the mentioned components. Although the terms “first”, “second”, or the like are used to describe various components, these components are not limited by these terms. These terms are only used to distinguish one component from another. Accordingly, it is to be understood that the first component referred to below may also be the second component within the technical concept of the present disclosure.

Unless otherwise defined, all terms (including technical and scientific terms) used in this specification may be used in the sense commonly understood by a person skilled in the art to which the present disclosure belongs. In addition, terms defined in commonly used dictionaries shall not be ideally or excessively interpreted unless explicitly specifically defined.

The terms “apparatus”, “unit”, or “module” used in this specification mean software, hardware components such as FPGAs or ASICs, and the “apparatus”, “unit”, or “module” performs certain roles. However, the “apparatus”, “unit”, or “module” is not limited to software or hardware. The “apparatus”, “unit”, or “module” may be configured to reside on an addressable storage medium and may be configured to cause one or more processors to execute. Thus, by way of example, the “apparatus”, “unit”, or “module” includes components such as software components, object-oriented software components, class components, and task components, as well as processes, functions, attributes, procedures, subroutines, segments of program code, drivers, firmware, microcode, circuitry, data, databases, data structures, tables, arrays, and variables. The functionality provided within the components and the “apparatus”, “unit”, or “module” may be combined into a smaller number of components and “apparatuses”, “units”, or “modules” or may be further separated into additional components and “apparatuses”, “units,” or “modules.

Spatially relative terms such as “below,” “beneath,” “lower,” “above,” “upper,” or the like may be used to easily describe the relationship between one component and other components as depicted in the drawings. Spatially relative terms should be understood to include different orientations of the components when used or operated in addition to the orientation depicted in the drawings. For example, when a component depicted in a drawing is flipped, a component described as “below” or “beneath” another component may be placed “above” the other component. Thus, the exemplary term “below” may include both the above and below directions. The components may also be oriented in other directions, and thus spatially relative terms may be interpreted according to the orientation.

In the present specification, a computer means any type of hardware device including at least one processor and may be understood to encompass software configurations operating on the hardware device according to an embodiment. For example, a computer may be understood to encompass, but is not limited to, a server, a smartphone, a tablet PC, a desktop, a laptop, and user clients and applications running on each device.

Hereinafter, an embodiment of the present disclosure will be described in detail with reference to the attached drawings.

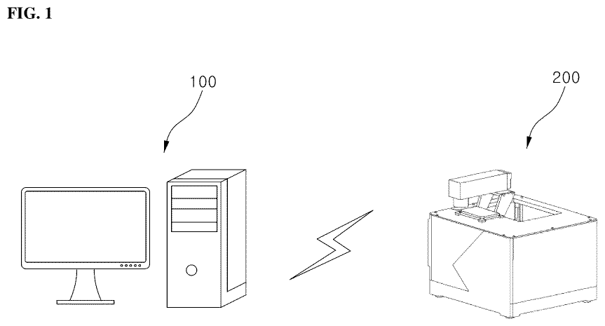

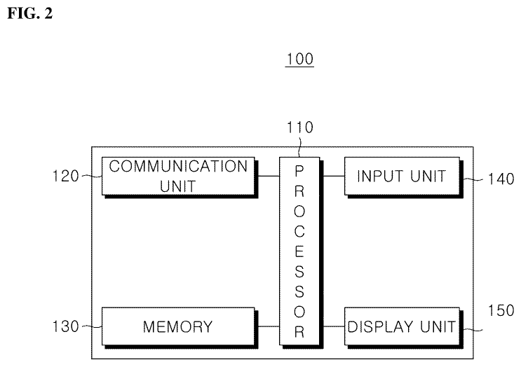

FIG. 1 is a connection configuration diagram between an apparatus for diagnosing an embryo implantation potential using an artificial intelligence-based diagnostic model and an embryo culture photographing device according to one embodiment of the present disclosure, and FIG. 2 is a block diagram of the apparatus for diagnosing an embryo implantation potential using an artificial intelligence-based diagnostic model according to one embodiment of the present disclosure.

Referring to FIG. 1 and FIG. 2, an apparatus 100 for diagnosing an embryo implantation potential using an artificial intelligence-based diagnostic model according to one embodiment of the present disclosure may input images generated by photographing one or more first embryos, which are determination targets of implantation potential index, as input data into a diagnostic model, and may receive implantation potential index data, which is a determination result of the implantation potential index for the first embryo from the diagnostic model, as output data.

In addition, the apparatus 100 for diagnosing an embryo implantation potential may further receive embryo position data, which is a result of detecting an embryo position, which is a position of an embryo image indicating the first embryo in an image, as output data from the diagnostic model.

To this end, the diagnostic model may be trained using training data to determine the implantation potential index and detect the embryo position.

The image input as input data to the diagnostic model for determining the implantation potential index and detecting the embryo position of the diagnostic model and the learning image used for training the diagnostic model may be generated by an embryo culture photographing device 200.

Specifically, the embryo culture photographing device 200 may generate an image by culturing one or more embryos and photographing the cultured embryos so that the embryos are included in one frame.

More specifically, the embryo culture photographing device 200 may generate the diagnostic image by culturing one or more first embryos that are the determination targets of the implantation potential index and photographing the cultured first embryos.

In addition, the embryo culture photographing device 200 may generate a learning image by culturing one or more second embryos used as samples and photographing the cultured second embryos.

Here, the embryo may be a preimplantation embryo, meaning an embryo from the time when a sperm and an oocyte are fertilized to the time before implantation.

To this end, the embryo culture photographing device 200 may be equipped with a culture chamber in which embryos are cultured, and a microscope photographing module for photographing embryos.

As long as the embryo culture photographing device 200 cultures embryos and photographs embryos, the shape and type thereof are not limited.

In this case, the apparatus 100 for diagnosing an embryo implantation potential using an artificial intelligence-based diagnostic model according to one embodiment of the present disclosure may obtain a diagnostic image and a learning image from the embryo culture photographing device 200 through wired or wireless communication.

Accordingly, the apparatus 100 for diagnosing an embryo implantation potential using an artificial intelligence-based diagnostic model according to one embodiment of the present disclosure may train the diagnostic model using the learning image, and obtain diagnostic ability index data for each first embryo using a diagnostic image.

To this end, the apparatus 100 for diagnosing an embryo implantation potential using an artificial intelligence-based diagnostic model according to one embodiment of the present disclosure may include a processor 110, a communication unit 120, a memory 130, an input unit 140, and a display unit 150.

Hereinafter, the process of the apparatus 100 for diagnosing an embryo implantation potential using an artificial intelligence-based diagnostic model according to one embodiment of the present disclosure training the diagnostic model and acquiring the diagnostic ability index data and embryo position data for each first embryo will be described.

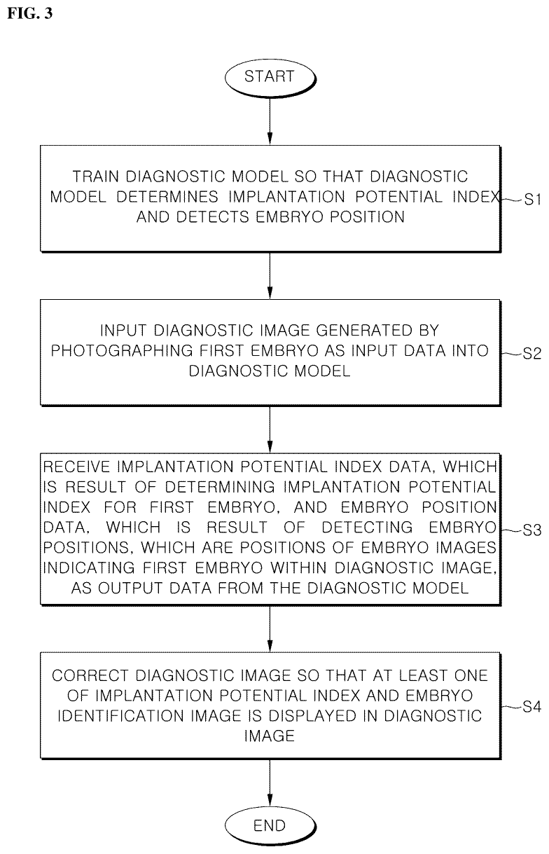

FIG. 3 is a flowchart of a method for diagnosing an embryo implantation potential using an artificial intelligence-based diagnostic model according to one embodiment of the present disclosure.

Referring to FIG. 3, the apparatus 100 for diagnosing an embryo implantation potential using an artificial intelligence-based diagnostic model according to one embodiment of the present disclosure may perform the method for diagnosing an embryo implantation potential using an artificial intelligence-based diagnostic model according to one embodiment of the present disclosure.

The processor 110 may train a diagnostic model M using training data so that the diagnostic model M determines the implantation potential index and detects the embryo position (S1).

To this end, the processor 110 may configure training data and train the diagnostic model M using the training data.

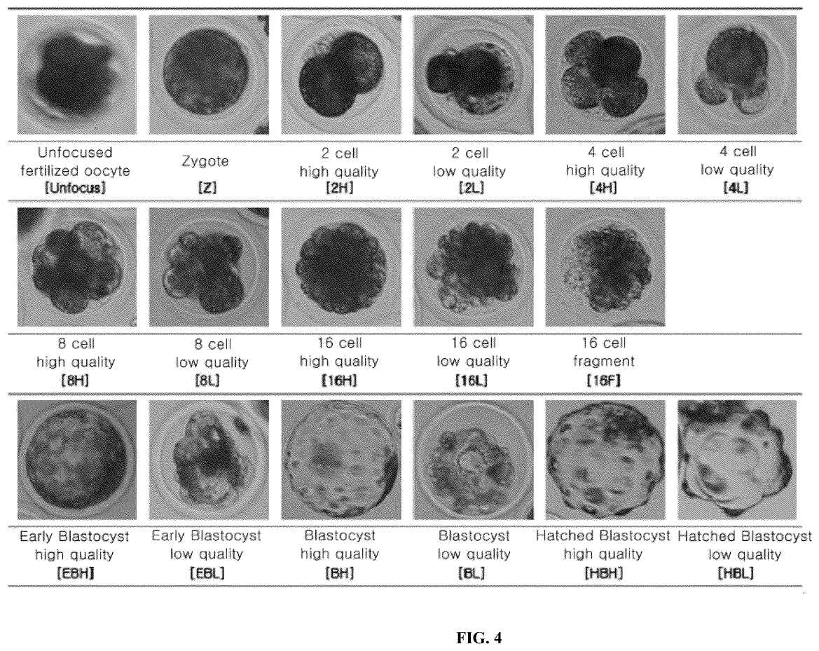

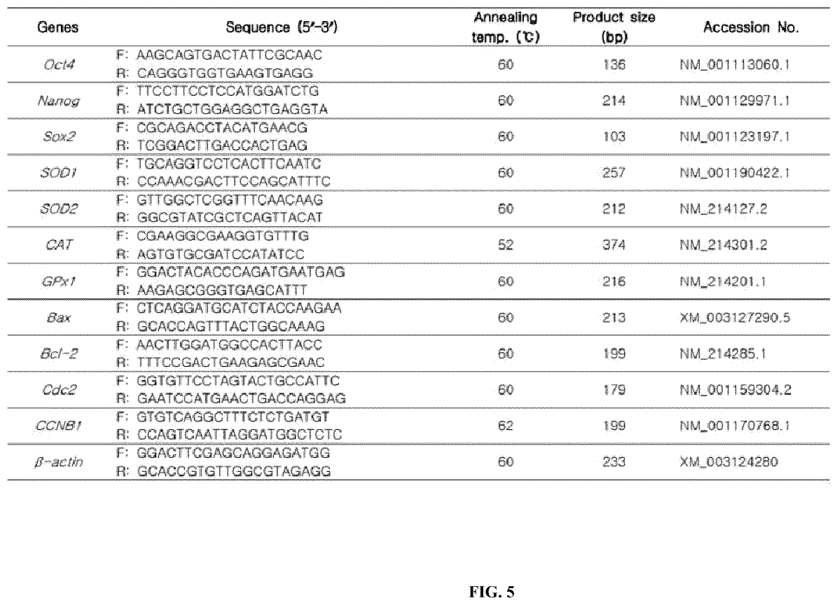

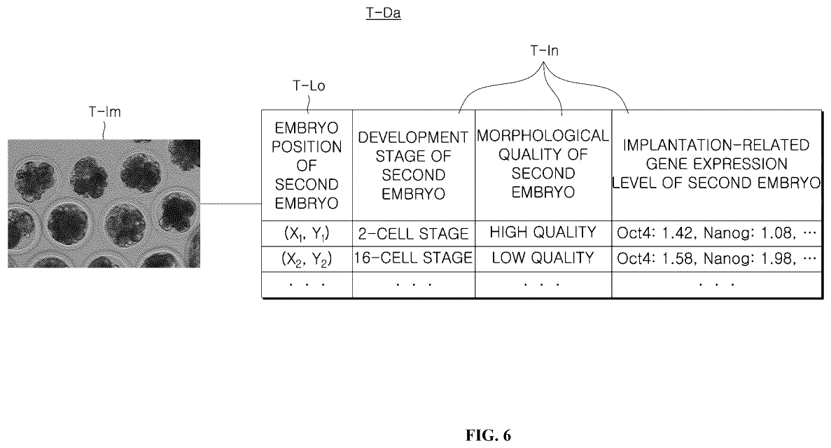

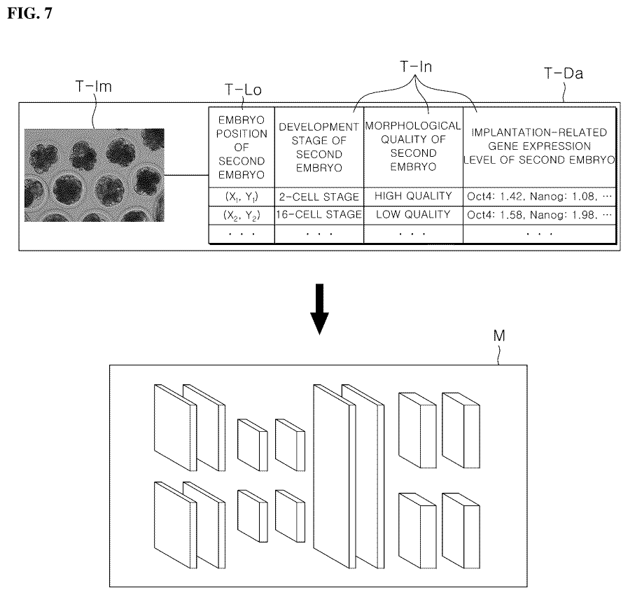

FIG. 4 is a diagram illustrating examples of a development stage of an embryo and a morphological quality of the embryo used in the apparatus and method for diagnosing an embryo implantation potential using an artificial intelligence-based diagnostic model according to one embodiment of the present disclosure, FIG. 5 is a diagram illustrating examples of embryo implantation-related genes used in the apparatus and method for diagnosing an embryo implantation potential using an artificial intelligence-based diagnostic model according to one embodiment of the present disclosure, FIG. 6 is a diagram illustrating examples of training data used in the apparatus and method for diagnosing an embryo implantation potential using an artificial intelligence-based diagnostic model according to one embodiment of the present disclosure, and FIG. 7 is a diagram for explaining a process of training a diagnostic model in the apparatus and method for diagnosing an embryo implantation potential and d using an artificial intelligence-based diagnostic model according to one embodiment of the present disclosure.

With further reference to FIGS. 4 to 7, the processor 110 may train the diagnostic model M to determine the implantation potential index for the embryo based on an image generated by photographing one or more embryos.

In addition, the processor 110 may train the diagnostic model M to determine an implantation potential index for the embryo based on the image generated by photographing one or more embryos.

The processor 110 may train the diagnostic model M to identify each embryo image indicating an embryo in the image generated by photographing one or more embryos and detect the embryo position which is a position of each embryo image.

To this end, the processor 110 may configure training data T-Da with a learning image T-Im generated by photographing one or more second embryos as samples and training implantation potential index data T-In, which is a result of pre-determining the implantation potential index for each second embryo.

Here, the training implantation potential index data T-In may be a result of pre-determining the implantation potential index for each second embryo by a researcher who confirmed the learning image T-Im.

Here, the implantation potential index may be an indicator indicating the implantation ability of each embryo.

Specifically, the implantation potential index may include one or more of the development stage of the embryo, the morphological quality of the embryo, and the implantation-related gene expression level of the embryo.

More specifically, the development stage of the embryo may be any one of a zygote stage, a 2-cell stage, a 4-cell stage, an 8-cell stage, a 16-cell stage, an early blastocyst stage, a blastocyst stage, and a hatched blastocyst stage.

Additionally, the development stage of the embryo may be an unfocused fertilized oocyte stage that is not in focus of the corresponding embryo image.

In this case, among the implantation potential indexes for each of the second embryos, the development stage may be the result of a researcher who confirmed the embryo image indicating the corresponding second embryo among the learning images pre-determining the embryo image as any one of the unfocused fertilized oocyte stage, the zygote stage, the 2-cell stage, the 4-cell stage, the 8-cell stage, the 16-cell stage, the early blastocyst stage, the blastocyst stage, and the hatched blastocyst stage.

Furthermore, the development stage may be obtained as an Unfocus value when the development stage among the implantation potential indexes for each of the second embryos is determined to be the unfocused fertilized oocyte stage, the development stage may be obtained as a Z value when the development stage among the implantation potential indexes for each of the second embryos is determined to be the zygote stage, the development stage may be obtained as a 2 value when the development stage among the implantation potential indexes for each of the second embryos is determined to be the 2-cell stage, the development stage may be obtained as a 4 value when the development stage among the implantation potential indexes for each of the second embryos is determined to be the 4-cell stage, the development stage may be obtained as an 8 value when the development stage among the implantation potential indexes for each of the second embryos is determined to be the 8-cell stage, the development stage may be obtained as a 16 value when the development stage among the implantation potential indexes for each of the second embryos is determined to be the 16-cell stage, the development stage may be obtained as an EB value when the development stage is determined to be the early blastocyst stage among the implantation potential indexes for each of the second embryos, the development stage may be obtained as a B value when the development stage is determined to be the blastocyst stage among the implantation potential indexes for each of the second embryos, and the development stage may be obtained as an HB value when the development stage is determined to be the hatched blastocyst stage among the implantation potential indexes for each of the second embryos.

Meanwhile, the morphological quality of the embryo may be any one of high quality, low quality, and fragmentation.

In this case, among the implantation potential indexes for each second embryo, the morphological quality may be the result of a researcher who confirmed the embryo image indicating the corresponding second embryo among the learning images and pre-determined as any one of the above-described high quality, low quality, and fragmentation.

Furthermore, the morphological quality may be obtained as an H value when the morphological quality among the implantation potential indexes for each of the second embryos is determined as the high quality, the morphological quality may be obtained as an L value when the morphological quality among the implantation potential indexes for each of the second embryos is determined as the low quality, and the morphological quality may be obtained as an F value when the morphological quality among the implantation potential indexes for each of the second embryos is determined as the fragmentation.

The implantation-related gene expression level of the embryo may indicate an expression level of genes involved in implantation during the process of implanting the embryo into the uterus.

Here, the genes involved in implantation during the process of implanting an embryo into the uterus may be one or more of the genes related to embryo pluripotency (Oct4, Nanog, Sox2), antioxidant capacity (SOD1, SOD2, CAT, GPx1), cell death (Bax, Bcl2), and cell division (Cdc2, CCNB1).

In this case, among the implantation potential indexes for each second embryo, the implantation-related gene expression level may be a value measured by a researcher extracting RNA from the second embryo, synthesizing cDNA, and performing PCR.

In addition, among the implantation potential indexes for each second embryo, the implantation-related gene expression level may be a relative value based on the value measured by a researcher extracting RNA from the second embryo, synthesizing cDNA, and performing PCR when the development stage of the second embryo was the zygote stage or the unfocused fertilized oocyte stage.

For example, when the development stage of the second embryo was the zygote stage or the unfocused fertilized oocyte stage, the value measured by a researcher extracting RNA from the second embryo, synthesizing cDNA, and performing PCR may be set as a standard 1.

In addition, the processor 110 may further add training embryo position data T-Lo, which is a result of pre-determining the embryo position of each embryo image indicating the second embryo within the learning image T-Im, to the training data T-Da, thereby configuring the training data T-Da.

Here, the training embryo position data T-Lo may be a result of pre-determining the embryo position of each embryo image indicating the second embryo within the learning image T-Im by a researcher who confirmed the learning image T-Im.

That is, the embryo position may be a coordinate indicating the position of the embryo or the area including the embryo in a coordinate system using the image as a coordinate plane.

In summary, the training implantation potential index data T-In and the training embryo position data T-Lo may be the results determined by the researcher directly confirming the learning image T-Im.

To this end, the communication unit 120 may be equipped with a communication module to receive at least one of the learning image T-Im, the training implantation potential index data T-In, and the training embryo position data T-Lo from the outside.

In addition, the input unit 140 may be equipped with an input module (for example, a mouse, a keyboard, or the like) to receive at least one of the training implantation potential index data T-In and the training embryo position data T-Lo.

In addition, the display unit 150 may display the learning image T-Im on the screen so that the researcher can confirm the learning image T-Im.

For this purpose, the display unit 150 may be equipped with a display module.

Thereafter, the processor 110 may configure the acquired learning image T-Im, training implantation potential index data T-In, and training embryo position data T-Lo as the training data T-Da as described above.

Then, the processor 110 may train the diagnostic model M to determine an implantation potential index for the embryo based on the image generated by photographing one or more embryos using the training data T-Da including the learning image T-Im, training implantation potential index data T-In, and training embryo position data T-Lo, and may train the diagnostic model M to identify each embryo image indicating the embryo in the image generated by photographing one or more embryos and detect the embryo position, which is the position of each embryo image.

In this case, the diagnostic model M may be stored in the memory 130.

Hereinafter, a process of obtaining diagnostic ability index data and embryo position data for each first embryo whose implantation potential index and embryo position have not been determined by the apparatus 100 for diagnosing an embryo implantation potential using an artificial intelligence-based diagnostic model according to one embodiment of the present disclosure will be described.

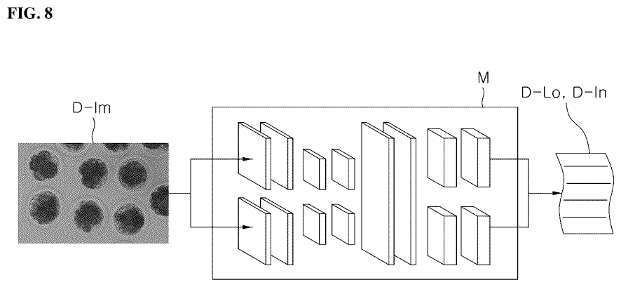

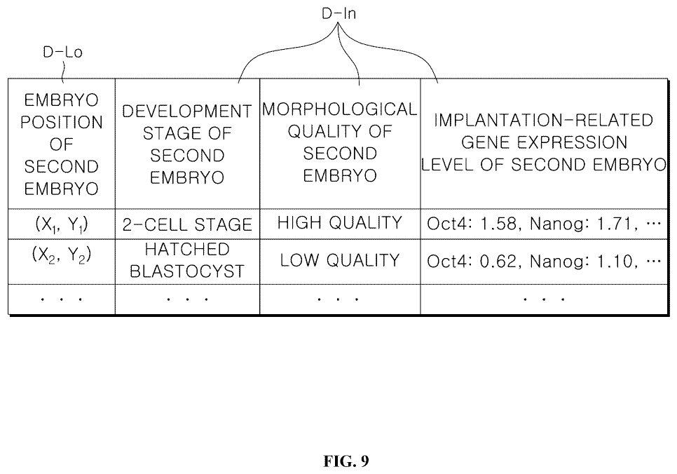

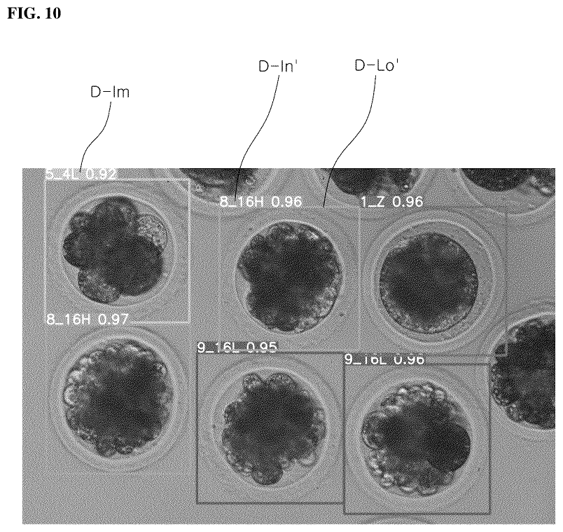

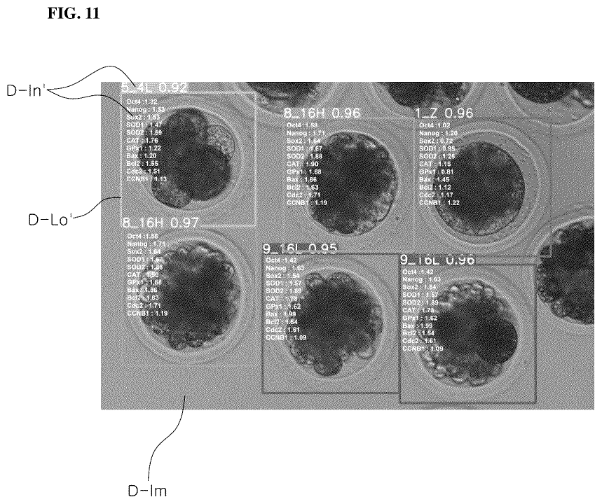

FIG. 8 is a diagram for explaining a process of acquiring diagnostic ability index data and embryo position data for each of the first embryos whose implantation potential index and embryo position are not determined in the apparatus and method for diagnosing an embryo implantation potential using an artificial intelligence-based diagnostic model according to one embodiment of the present disclosure, FIG. 9 is a diagram illustrating examples of the diagnostic ability index data and embryo position data acquired by the apparatus and method for diagnosing an embryo implantation potential using an artificial intelligence-based diagnostic model according to one embodiment of the present disclosure, FIG. 10 is an example of a diagnostic image corrected to display an embryo identification image and an implantation potential index by the apparatus and method for diagnosing an embryo implantation potential using an artificial intelligence-based diagnostic model according to one embodiment of the present disclosure, and FIG. 11 is another example of the diagnostic image corrected to display the embryo identification image and the implantation potential index by the apparatus and method for diagnosing an embryo implantation potential using an artificial intelligence-based diagnostic model according to one embodiment of the present disclosure.

Referring further to FIGS. 8 to 11, the processor 110 may input a diagnostic image D-Im generated by photographing one or more first embryos as determination targets of the implantation potential index as input data into the diagnostic model M (S2), and receive the implantation potential index data D-In, which is the result of determining the implantation potential index for the first embryo, as output data from the diagnostic model M (S3).

In addition, the processor 110 may input the diagnostic image D-Im as input data to the diagnostic model M, and further receive embryo position data D-Lo, which is a result of detecting embryo positions, which are positions of embryo images indicating the first embryo within the diagnostic image D-Im, as output data from the diagnostic model M.

Here, the implantation potential index data D-In is data indicating an index with the same meaning as the training implantation potential index data T-In, but the only difference is that the target of the index is the first embryo.

In addition, the embryo position data D-Lo is data indicating coordinates with the same meaning as training embryo position data T-Lo, but the only difference is that the target of the coordinates is the first embryo.

Thereafter, when implantation potential index data D-In and embryo position data D-Lo are output from the diagnostic model M, the processor 110 may correct the diagnostic image D-Im so that at least one of the implantation potential index data D-In and the embryo position data D-Lo is displayed in the diagnostic image D-Im (S4).

To this end, the processor 110 may set the identification image position, which is the position at which an embryo identification image D-Lo′ for identifying the embryo image indicating the first embryo in the diagnostic image D-Im is displayed in the diagnostic image D-Im, based on the embryo position indicated by the embryo position data D-Lo.

Specifically, the processor 110 may set an identification image position to coordinates of a rectangular area so that the embryo image indicating the corresponding first embryo is included in the embryo identification image D-Lo′. To this end, the processor 110 may set the coordinates of the upper left corner of the rectangular area and the coordinates of the lower right corner of the rectangular area as the identification image position.

Thereafter, the processor 110 may correct the diagnostic image D-Im so that the embryo identification image D-Lo′ is displayed at the identification image position.

Here, the embryo identification image D-Lo′ may be a rectangular box.

Meanwhile, the processor 110 may set an index display position, which is the position at which the implantation potential index indicated by the implantation potential index data D-In is displayed within the diagnostic image D-Im, based on the embryo position indicated by the embryo position data D-Lo.

Specifically, the processor 110 may set the index display position so that a development stage D-In′ among the implantation potential indexes indicated by the implantation potential index data D-In is displayed at the uppermost end of the embryo identification image D-Lo′ corresponding to the first embryo, and a morphological quality D-In′ among the implantation potential indexes indicated by the implantation potential index data D-In is displayed to the right of the development stage D-In′ among the uppermost ends of the embryo identification image D-Lo′ corresponding to the first embryo.

In addition, the processor 110 may set an index display position so that the implantation-related gene expression level D-In′ among the implantation potential indexes indicated by the implantation potential index data D-In is displayed on the inside of the embryo identification image D-Lo′ corresponding to the first embryo.

In other words, the processor 110 may set the index display position so that the implantation-related gene expression level D-In′ among the implantation potential indexes indicated by the implantation potential index data D-In is displayed in an overlapping manner in the embryo image indicating the first embryo.

Through this, the processor 110 may correct the diagnostic image D-Im so that the embryo identification image D-Lo′ that identifies the corresponding embryo image and the implantation potential index D-In′ that indicates the implantation potential index data D-In of the corresponding first embryo are displayed together with each embryo image of the first embryo.

In this case, the processor 110 according to another embodiment may calculate a separation distance between the plurality of index display positions corresponding to each of the plurality of first embryos, and when there are two index display positions whose separation distance is less than a standard separation distance, the two index display positions may be corrected so that the separation distance between the two index display positions whose separation distance is less than the standard separation distance is equal to or more than the standard separation distance. Through this, the processor 110 can minimize the overlap between the implantation potential index D-In′, such as the development stage D-In′, the quality D-In′, and the implantation-related gene expression level D-In′ within the corrected diagnostic image D-Im.

Finally, the processor 110 may control the display unit 150 so that the corrected diagnostic image D-Im is displayed.

Accordingly, the researcher can easily check the implantation potential index for each first embryo through the corrected diagnostic image D-Im.

Meanwhile, the processor 110 may control the overall operation of the apparatus 100 for diagnosing an embryo implantation potential using an artificial intelligence-based diagnostic model by using various programs stored in the memory 130. The processor 110 may include a RAM, a ROM, a graphic processing unit, a main CPU, the first to n interfaces, and a bus. In this case, the RAM, the ROM, the graphic processing unit, the main CPU, the first to n interfaces or the like may be connected to each other through a bus.

The RAM stores O/S and application programs. Specifically, when the apparatus 100 for diagnosing an embryo implantation potential using an artificial intelligence-based diagnostic model is booted, the O/S is stored in the RAM, and various application data selected by the user may be stored in the RAM.

The ROM stores a command set for system booting, and the like. When a turn-on command is input and power is supplied, the main CPU copies the O/S stored in the memory 130 to the RAM according to the command stored in the ROM, and executes the O/S to boot the system. When booting is completed, the main CPU copies various application programs stored in the memory 130 to the RAM, and executes the application programs copied to the RAM to perform various operations.

The main CPU accesses the memory 130 and performs booting using the OS stored in the memory 130. Then, the main CPU performs various operations using various programs, contents, data, and the like stored in the memory 130.

The first to nth interfaces are connected to the various components described above. One of the first to n interfaces may be a network interface that is connected to an external device via a network.

Meanwhile, furthermore, the processor 110 may control the artificial intelligence model. In this case, the processor 110 may include a graphics-only processor (for example, GPU) for controlling the artificial intelligence model.

Meanwhile, the artificial intelligence model M which an artificial intelligence model according to the present disclosure may use a decoder based on a transformer model that overcomes the long-term dependency limitations of a multi-view encoder and a recurrent neural network to obtain relationship information between features of an input image.

Unlike the traditional method using a recurrent neural network, the artificial intelligence model M which is the artificial intelligence model according to the present disclosure may use the structure of a transformer model, which is a machine translation model trained only with an attention mechanism technique.

The transformer model included in the artificial intelligence model M which the artificial intelligence model according to the present disclosure may be implemented using an encoder and a decoder. For example, the transformer model may be implemented by one or more encoders and one or more decoders.

Meanwhile, the diagnostic model M, which is an artificial intelligence model according to the present disclosure, may be a model based on supervised learning or unsupervised learning. Furthermore, the diagnostic model M, which is an artificial intelligence model according to the present disclosure, may include a support vector machine (SVM), a decision tree, a neural network, and the like, and methodologies applied thereto.

As an example, the diagnostic model M, which is an artificial intelligence model according to the present disclosure, may be an artificial intelligence model based on a convolutional deep neural network (CNN) trained by inputting training data. However, the present disclosure is not limited thereto, and it goes without saying that various artificial intelligence models may be applied to the present disclosure. For example, models such as a deep neural network (DNN), a recurrent neural network (RNN), and a bidirectional recurrent deep neural network (BRDNN) may be used as artificial intelligence models, but are not limited thereto.

In this case, convolutional deep neural networks (CNNs) are a type of multilayer perceptrons designed to use minimal preprocessing. The convolutional neural networks include one or more convolutional layers and general artificial neural network layers placed on the layers, and additionally utilize weight and pooling layers. Thanks to this structure, convolutional neural networks may fully utilize two-dimensional structured input data. In addition, convolutional neural networks may be trained through standard backpropagation. The convolutional neural networks have the advantage of being easier to train than other feedforward artificial neural network techniques and using fewer parameters.

In addition, the deep neural networks (DNNs) are artificial neural networks (ANNs) including multiple hidden layers between the input layer and the output layer.

In this case, the structure of the deep neural networks may include a perceptron. The perceptron includes multiple input values, one processor, and one output value. The processor multiplies various input values by each weight, and then adds all the input values that have been multiplied by the weights. Then, the processor inputs the added value into the activation function and outputs one output value. When a specific value is desired to be output as the activation function output value, the weight multiplied by each input value may be corrected and the output value may be recalculated using the corrected weight. In this case, each perceptron may use a different activation function. In addition, each perceptron receives the outputs transmitted from the previous layer as input and then uses the activation function to obtain the output. The obtained output is transmitted as the input of the next layer. After going through the process described above, finally, it is possible to obtain several output values.

A recurrent neural network (RNN) is a neural network in which the connections between the units that constitute the artificial neural network form a directed cycle. Unlike a feed-forward neural network, the recurrent neural network may utilize the memory within the neural network to process an arbitrary input.

Deep belief networks (DBN) are a generative graphical model used in machine learning, and in deep learning, the deep belie networks refer to deep neural networks consisting of multiple layers of latent variables. The deep belief networks have the characteristic that there are connections between layers, but no connections between units within the layers.

The deep belief networks can be used for pre-learning due to their generative nature, and after learning the initial weights through pre-learning, the weights may be fine-tuned through backpropagation or other discriminant algorithms. The characteristics are very useful when there is little training data, because the smaller the training data, the greater the influence of the initial weight values on the resulting model. The initial weight values that have been pre-learned are closer to the optimal weights than the initial weight values that are set arbitrarily, which enables improved performance and speed in the fine-tuning stage.

The above-described artificial intelligence and its learning method are described for illustrative purposes, and the artificial intelligence and its learning method used in the above-described embodiments are not limited. For example, all types of artificial intelligence technologies and their learning methods that can be applied by a person skilled in the art to solve the same problem can be utilized to implement the system according to the disclosed embodiment.

Meanwhile, the processor 110 may include one or more cores (not illustrated) and a graphics processing unit (not illustrated) and/or a connection path (for example, a bus, and the like) for transmitting and receiving signals with other components.

The processor 110 according to one embodiment performs the method described in relation to the present disclosure by executing one or more instructions stored in the memory 130.

For example, the processor 110 may acquire new training data by executing one or more instructions stored in the memory 130, perform a test on the acquired new training data using the trained model, extract first training data in which labeled information is acquired with an accuracy higher than a predetermined first reference value as a result of the test, delete the extracted first training data from the new training data, and re-learn the trained model using the new training data from which the extracted training data has been deleted.

Meanwhile, the processor 110 may further include a random-access memory (RAM) (not illustrated) and a read-only memory (ROM) (not illustrated) that temporarily and/or permanently store signals (or data) processed within the processor 110. In addition, the processor 110 may be implemented in the form of a system on chip (SoC) including at least one of a graphic processing unit, a RAM, and a ROM.

The memory 130 may store programs (one or more instructions) for processing and controlling the processor 110. The programs stored in the memory 130 may be divided into multiple modules according to their functions.

The memory 130 may store various programs and data required for the operation of the apparatus 100 for diagnosing an embryo implantation potential using an artificial intelligence-based diagnostic model. The memory 130 may be implemented as a nonvolatile memory 130, a volatile memory 130, a flash memory 130, a hard disk drive (HDD), or a solid-state drive (SSD).

The communication unit 120 may perform communication. In particular, the communication unit 120 may include various communication chips such as a Wi-Fi chip, a Bluetooth chip, a wireless communication chip, an NFC chip, a low-power Bluetooth chip (BLE chip), and the like. In this case, the Wi-Fi chip, the Bluetooth chip, and the NFC chip perform communication in a LAN mode, a Wi-Fi mode, a Bluetooth mode, and an NFC mode, respectively. In the case of using a Wi-Fi chip or a Bluetooth chip, various connection information such as SSID and session key are first transmitted and received, and then communication is established using these, and then various information can be transmitted and received. A wireless communication chip refers to a chip that performs communication according to various communication standards such as IEEE, Zigbee, 3rd Generation (3G), 3rd Generation Partnership Project (3GPP), Long Term Evolution (LTE), 5th Generation (5G), and the like.

The steps of the method or algorithm described in relation to the embodiments of the present disclosure may be implemented directly in hardware, implemented as a software module executed by hardware, or implemented by a combination of these. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or any form of computer-readable recording medium well known in the technical field to which the present disclosure belongs.

In addition, the different embodiments of the present disclosure may be complementary or combined.

The components of the present disclosure may be implemented as a program (or application) to be executed in combination with a computer as hardware and stored on a medium. The components of the present disclosure may be executed as software programming or software elements, and similarly, the embodiments may be implemented in a programming or scripting language such as C, C++, Java, assembler, Python, and the like, including various algorithms implemented as a combination of data structures, processes, routines, or other programming components. The functional aspects may be implemented as an algorithm executed on one or more processors.

Although the embodiments of the present disclosure have been described with reference to the attached drawings, those skilled in the art will understand that the present disclosure may be implemented in other specific forms without changing the technical idea or essential features thereof. Therefore, the embodiments described above should be understood as illustrative in all respects and not restrictive.

What is claimed is:

1. An apparatus for diagnosing an embryo implantation potential using an artificial intelligence-based diagnostic model, the apparatus comprising:
a memory storing a diagnostic model trained to determine an implantation potential index for an embryo based on an image generated by photographing one or more embryos formed by fertilization of a sperm and an oocyte and before implantation; and
a processor for inputting a diagnostic image generated by photographing one or more first embryos, which are determination targets of the implantation potential index, into the diagnostic model as input data, and receiving implantation potential index data, which is a result of determining the implantation potential index for the first embryo, as output data from the diagnostic model.

2. The apparatus of claim 1, wherein the diagnostic model is trained using training data including a learning image generated by photographing one or more second embryos as samples and training implantation potential index data, which is a result of pre-determining the implantation potential index for the second embryo.

3. The apparatus of claim 1, wherein the implantation potential index includes one or more of a development stage of the embryo, a morphological quality of the embryo, and an implantation-related gene expression level of the embryo.

4. The apparatus of claim 1, wherein the diagnostic model is trained to identify an embryo image indicating the embryo in the image and detect an embryo position which is a position of the embryo image, and
the processor inputs the diagnostic image generated by photographing the first embryo into the diagnostic model as input data, and receives embryo position data, which is s result of detecting an embryo position, which is a position of the embryo image indicating the first embryo in the diagnostic image, from the diagnostic model as output data.

5. The apparatus of claim 4, wherein the processor
sets an index display position where the implantation potential index indicated by the implantation potential index data is a position displayed in the diagnostic image, based on the embryo position indicated by the embryo position data, and
corrects the diagnostic image so that the implantation potential index is displayed at the index display position.

6. The apparatus of claim 4, wherein the processor
sets an embryo identification image position which is a position where an embryo identification image for identifying the embryo image indicating the first embryo image in the diagnostic image is displayed in the diagnostic image, based on the embryo position indicated by the embryo position data, and
corrects the diagnostic image so that the embryo identification image is displayed at the embryo identification image position.

7. A method for diagnosing an embryo implantation potential using an artificial intelligence-based diagnostic model, the method comprising:
storing, in a memory, a diagnostic model trained to determine an implantation potential index for an embryo based on an image generated by photographing one or more embryos formed by fertilization of a sperm and an oocyte and before implantation; and
inputting a diagnostic image generated by photographing one or more first embryos, which are determination targets of the implantation potential index, into the diagnostic model as input data, and receiving implantation potential index data, which is a result of determining the implantation potential index for the first embryo, as output data from the diagnostic model, using a processor.

8. The method of claim 7, further comprising training the diagnostic model using training data including a learning image generated by photographing one or more second embryos as samples and training implantation potential index data, which is a result of pre-determining the implantation potential index for the second embryo.

9. The method of claim 7, wherein the implantation potential index includes one or more of a development stage of the embryo, a morphological quality of the embryo, and an implantation-related gene expression level of the embryo.

10. The method of claim 7, further comprising:
training the diagnostic model to identify an embryo image indicating the embryo in the image and detect an embryo position which is a position of the embryo image; and
inputting the diagnostic image generated by photographing the first embryo into the diagnostic model as input data, and receiving embryo position data, which is s result of detecting an embryo position, which is a position of the embryo image indicating the first embryo in the diagnostic image, from the diagnostic model as output data, using the processor.

11. The method of claim 10, further comprising:
setting, using the processor, an index display position where the implantation potential index indicated by the implantation potential index data is a position displayed in the diagnostic image, based on the embryo position indicated by the embryo position data; and
correcting, using the processor, the diagnostic image so that the implantation potential index is displayed at the index display position.

12. The method of claim 10, further comprising:
setting, using the processor, an embryo identification image position which is a position where an embryo identification image for identifying the embryo image indicating the first embryo image in the diagnostic image is displayed in the diagnostic image, based on the embryo position indicated by the embryo position data; and
correcting, using the processor, the diagnostic image so that the embryo identification image is displayed at the embryo identification image position.
